(19) United States
(12) Patent Application Publication
(10) Pub. No.: US 2023/0404884 A1
(43) Pub. Date: Dec. 21, 2023

(54) COSMETIC COMPOSITION COMPRISING HYDROPHILIC ANTIOXIDANTS AND THIOPYRIDINONE COMPOUNDS

(71) Applicant: L'OREAL, Paris (FR)

(72) Inventors: Nicholas David STEBBINS, Rahway, NJ (US); David CHAN, Oradell, NJ (US)

(73) Assignee: L'OREAL, Paris (FR)

(21) Appl. No.: 17/844,939

(22) Filed: Jun. 21, 2022

(51) Int. Cl. A61K 8/49; A61K 8/67; A61Q 19/02

(52) U.S. Cl. CPC A61K 8/4933; A61K 8/676; A61Q 19/02

(57) ABSTRACT

The instant disclosure relates to cosmetic compositions comprising: (i) compounds of Thiopyridinone type; (ii) one or more hydrophilic antioxidants; and wherein the compound of thiopyridinone type and the one or more hydrophilic antioxydants are present in the composition in amounts sufficient to produce stability of (i) and (ii).

### FIELD OF THE INVENTION

The present disclosure relates to cosmetic compositions comprising a combination of ascorbic acid and its derivatives with compounds of Thiopyridinone type.

### BACKGROUND OF THE INVENTION

Vitamin C is a potent antioxidant that can be used topically in dermatology to treat and prevent changes associated with photoageing, hyperpigmentation, etc. Vitamin C is very unstable and difficult to formulate and deliver into the dermis. Research has been ongoing to develop stable compositions that deliver high amounts of ascorbic acid to the skin.

### SUMMARY OF THE DISCLOSURE

The present disclosure relates to cosmetic compositions that are unique in comparison to conventional cosmetic compositions because they contain an association of two molecules, compounds of Thiopyridinone type (I) or (I′) and hydrophilic antioxidants, that show a co-stabilization effect.

The inventors have discovered that combining compounds of Thiopyridinone type and hydrophilic antioxidants improves the stability of both.

The cosmetic compositions typically include:
i) at least one compound selected from compounds of formula (I) and tautomer of formula (I′) herein below; and their optical isomers, racemates, and/or solvates such as hydrates, alone or as a mixture:

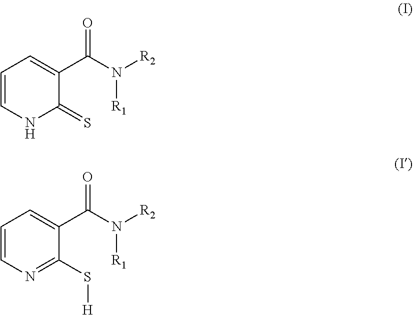

In which Formulas (I) and (I′):
   R₁ denotes a radical chosen from:
      a) a hydrogen atom;
      b) a saturated linear C₁-C₁₀ or branched C₃-C₁₀ alkyl group optionally substituted with one or more groups, which may be identical or different, chosen from:
      i)—O—R₃
      ii)—S—R₃;
   R₂ denotes a radical chosen from:
      a) a hydrogen atom;
      b) a saturated hydrocarbonated group linear C₁-C₁₂ or branched C₃-C₁₂ or cyclic C₃-C₈, optionally substituted with one or more groups, which may be identical or different, chosen from:
      i)—O—R₃
      ii)—S—R₃
      iii)—C(O)—O—R₃;
      iv) a C₅-C₁₂ aryl group optionally substituted with one or more hydroxyls and/or with one or more C₁-C₈ alkoxy radicals;
      c) a C₅-C₁₂ aryl group optionally substituted with one or more hydroxyls and/or with one or more C₁-C₈ alkoxy radicals
   R₃ denotes a radical chosen from:
a) a hydrogen atom;
b) a saturated linear C₁-C₁₀ or branched C₃-C₁₀ alkyl group;
ii) one or more hydrophilic antioxidants.

The inventors surprisingly and unexpectedly discovered that when compounds of thiopyridinone type and one or more ascorbic hydrophilic antioxidants are present in the composition in amounts sufficient, the association produced a co-stability of the compounds of thiopyridinone type and the one or more hydrophilic antioxidants.

In some embodiments, the one or more hydrophilic antioxidants is typically present in the cosmetic composition in an amount of from about 0.1 to about 20 wt. %, based on the total weight of the cosmetic composition. In some cases, the amount of hydrophilic antioxidants is higher than 5 wt. %, for example from about 6 wt. % to about 15 wt. %, from about 8 wt. % to about 15 wt. %, from about 9 wt. % to about 15 wt. %, or from about 10 wt. % to about 15 wt. %, based on the total weight of the composition. In one embodiment, the one or more hydrophilic antioxidants is present from about 1 to about 15 wt. %, based on the total weight of the cosmetic composition. In one embodiment, the one or more hydrophilic antioxidants is preferably chosen from acid ascorbic and its derivatives.

In some embodiments, the compound i) is typically present in the cosmetic compositions in an amount of from about 0.01 to about 10 wt. %, based on the total weight of the cosmetic composition. In some cases, the amount of compound i) is present in an amount of from about 0.1 to about 5 wt. %, based on the total weight of the cosmetic composition. In some embodiments, the compound i) is typically present in the cosmetic compositions in an amount of from about 0.5 to about 3 wt. %, based on the total weight of the cosmetic composition

In some embodiments, the cosmetic compositions may include one or more fatty compounds.

In one or more embodiments, the cosmetic compositions may include one or more polymers.

In some embodiments, the cosmetic compositions may include one or more emulsifiers.

In one or more embodiments, the cosmetic composition may have a pH ranging from about 4.5 to about 6.5, preferably from about 5 to about 6.

In some embodiments, the cosmetically acceptable carrier system is selected from oil-in-water emulsions, water-in-oil emulsions, silicone in water emulsions, dispersions, micellar waters, biphasic compositions, foams, and alcohol-based carriers.

In some embodiments, the cosmetic compositions may include:
i) from about 0.01 to about 10 wt. % of at least one compound selected from compounds of formula (I) and tautomer of formula (I′) herein below; and their optical isomers, racemates, and/or solvates such as hydrates, alone or as a mixture:

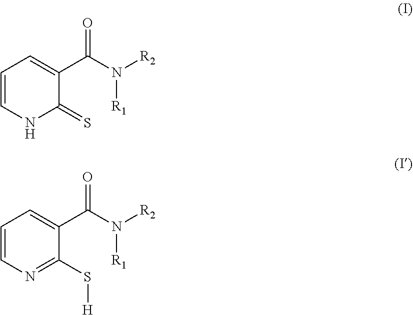

In which Formulas (I) and (I′):
   R₁ denotes a radical chosen from:
      a) a hydrogen atom;
      b) a saturated linear C₁-C₁₀ or branched C₃-C₁₀ alkyl group optionally substituted with one or more groups, which may be identical or different, chosen from:
      i)—O—R₃
      ii)—S—R₃;
   R₂ denotes a radical chosen from:
      a) a hydrogen atom;
      b) a saturated hydrocarbonated group linear C₁-C₁₂ or branched C₃-C₁₂ or cyclic C₃-C₈, optionally substituted with one or more groups, which may be identical or different, chosen from:
      i)—O—R₃
      ii)—S—R₃
      iii)—C(O)—O—R₃;
      iv) a C₅-C₁₂ aryl group optionally substituted with one or more hydroxyls and/or with one or more C₁-C₈ alkoxy radicals;
      c) a C₅-C₁₂ aryl group optionally substituted with one or more hydroxyls and/or with one or more C₁-C₈ alkoxy radicals
   R₃ denotes a radical chosen from:
a) a hydrogen atom;
b) a saturated linear C₁-C₁₀ or branched C₃-C₁₀ alkyl group;
ii) from about 0.1 to about 20 wt. % of one or more ascorbic acid and its derivatives; and
iii) a cosmetic suitable carrier;
wherein the i) compound of thiopyridininone type and the ii) one or more ascorbic acid and its derivatives are present in the composition in amounts sufficient to produce stability of i) and ii); and wherein the weight percentages are based on the total weight of the cosmetic composition.

The instant disclosure relates to methods for co-stabilizing compound of thiopyridinone type (I) or (I′) and hydrophilic antioxidants when both are present in the composition in amount sufficient to produce stability of compound of thiopyridinone type and hydrophilic antioxidants. The cosmetic compositions are useful for treating skin, for examples, the skin of the face and neck of a human. Thus, the instant disclosure relates to methods of treating the skin comprising application of the cosmetic composition of the instant disclosure to the skin. The cosmetic compositions are additionally useful in methods for treating dark spots, and uneven skin texture. The aforementioned methods may be non-therapeutic.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE DISCLOSURE

The present disclosure relates to cosmetic compositions typically including at least one compound corresponding to the following formula (I) or (I′), referred to as “compound of thiopyridinone type”.

The present disclosure provides cosmetic compositions that are unique in comparison to conventional cosmetic compositions because they contain an association of two molecules, compound of thiopyridinone type (I) or (I′) and hydrophilic antioxidants, that show a co-stabilization effect.

The inventors have discovered that combining the two molecules improves the stability of both.

The cosmetic compositions of the instant disclosure typically include:
i) at least one compound selected from compounds of formula (I) and tautomer of formula (I′) herein below; and their optical isomers, racemates, and/or solvates such as hydrates, alone or as a mixture:

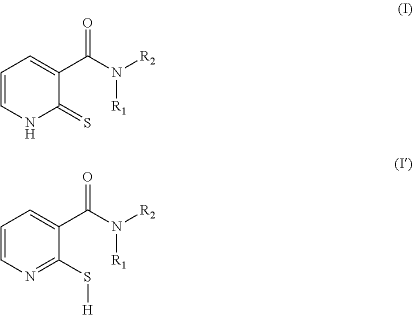

In which Formulas (I) and (I′):
   R₁ denotes a radical chosen from:
      a) a hydrogen atom;
      b) a saturated linear C₁-C₁₀ or branched C₃-C₁₀ alkyl group optionally substituted with one or more groups, which may be identical or different, chosen from:
      i)—O—R₃
      ii)—S—R₃;
   R₂ denotes a radical chosen from:
      a) a hydrogen atom;
      b) a saturated hydrocarbonated group linear C₁-C₁₂ or branched C₃-C₁₂ or cyclic C₃-C₈, optionally substituted with one or more groups, which may be identical or different, chosen from:
      i)—O—R₃
      ii)—S—R₃;
      iii)—C(O)—O—R₃;
      iv) a C₅-C₁₂ aryl group optionally substituted with one or more hydroxyls and/or with one or more C₁-C₈ alkoxy radicals;
      c) a C₅-C₁₂ aryl group optionally substituted with one or more hydroxyls and/or with one or more C₁-C₈ alkoxy radicals
   R₃ denotes a radical chosen from:
a) a hydrogen atom;
b) a saturated linear C₁-C₁₀ or branched C₃-C₁₀ alkyl group;
ii) one or more ascorbic acid and its derivatives.

Applicants formulated a novel association of compound of thiopyridinone type with one or more hydrophilic antioxidants that demonstrates an unexpected co-stability of compound of thiopyridinone type and the one or more hydrophilic antioxidants.

Cosmetic formulations comprising the novel association that are appropriate for topical application to the skin include compositions in the form of a lotion, serum, gel, milk, foam, liquid foundation or cream.

A suitable cosmetically acceptable carrier system according to the disclosure may be selected from any one of oil-in-water emulsions, water-in-oil emulsions, silicone in water emulsions, dispersions, micellar waters, biphasic compositions, foams, and alcohol based.

### Compounds of Thiopyridinone Type

For the purposes of the instant disclosure, and unless otherwise indicated:
a “saturated hydrocarbonated group linear C₁-C₁₂ or branched C₃-C₁₂” is equivalent to a “linear (C₁-C₁₂)alkyl or branched (C₃-C₁₂)alkyl group” which correspond to a saturated C₁-C₁₂ linear or branched C₃-C₁₂ hydrocarbon based group, and preferably C₁-C₁₀ linear or C₃-C₁₀ branched hydrocarbon based group, more preferably C₁-C₆ linear or C₃-C₆ branched hydrocarbon-based; Preferentially, the linear or branched groups may be chosen from methyl, ethyl, propyl, isopropyl, butyl, isobutyl and tert-butyl, pentyl, hexyl, heptyl, octyl, nonyl and decyl.
More preferentially, the saturated linear or branched alkyl groups may be chosen from methyl, ethyl, propyl, isopropyl, butyl, isobutyl and tert-butyl, pentyl, hexyl, heptyl and octyl, such as methyl, ethyl, n-pentyl, n-nonyl, isobutyl.
a saturated hydrocarbonated cyclic C₃-C₈ group is a mono or bicyclic cycloalkyl group containing from 3 to 8 carbon atoms especially is a monocyclic cycloalkyl group in C₅ to C₇ such as cyclohexyl group,
an “alkoxy radical” is an alkyl-oxy radical for which the alkyl radical is a linear or branched C₁-C₁₆ and preferentially C₁-C₈ hydrocarbon-based radical;
when the alkoxy group is optionally substituted, this implies that the alkyl group is optionally substituted as defined hereinabove;
an “aryl” group represents a fused or non-fused monocyclic or bicyclic carbon-based group comprising from 5 to 12 carbon atoms, preferably from 6 to 10 carbon atoms, and in which at least one ring is aromatic; preferentially, the aryl radical is a phenyl, biphenyl, naphthyl, more preferably a phenyl group;
the term “at least one” is equivalent to the term “one or more”; and
the term “inclusive” for a range of concentrations means that the limits of that range are included in the defined range.
   The salts of the compounds of formula (I), (I′), (II) or (II′) as defined herein after comprise the conventional non-toxic salts of said compounds, such as those formed from organic or inorganic acid or from organic or inorganic base.
   As salts of the compounds of formula (I), (I′), (II) or (II′) mention may be made of: the salts obtained by addition of the compound of formula (I) or (II) to:
a mineral base, such as sodium hydroxide, potassium hydroxide, calcium hydroxide, ammonium hydroxide, magnesium hydroxide, lithium hydroxide, and sodium, potassium or calcium carbonate or hydrogen carbonate for example;
   or
an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made in particular of 2-amino-2-methylpropanol, ethanolamine, triethanolamine, 2-dimethylaminopropanol, 2-amino-2-(hydroxymethyl)-1,3-propanediol and 3-(dimethylamino)propylamine.
   Mention may also be made of the salts of amino acids, for instance lysine, arginine, guanidine, glutamic acid and aspartic acid. Advantageously, the salts of the compounds of formula (I) or (II) (when it comprises a carboxy group) may be chosen from alkali metal or alkaline-earth metal salts such as sodium, potassium, calcium or magnesium salts and ammonium salts.
as “organic or inorganic acid salt” is more particularly chosen from salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
The acceptable solvates of the compounds described in the instant disclosure comprise conventional solvates such as those formed during the preparation of said compounds owing to the presence of solvents. Mention may be made, by way of example, of the solvates due to the presence of water or of linear or branched alcohols, such as ethanol or isopropanol.
The optical isomers are in particular, the enantiomers and the diastereoisomers.
   The compounds used according to the instant disclosure therefore correspond to formula (I) or tautomer (I′) below or their salts, their optical isomers, racemates, and/or solvates such as hydrates and the thereof, alone or as a mixture.

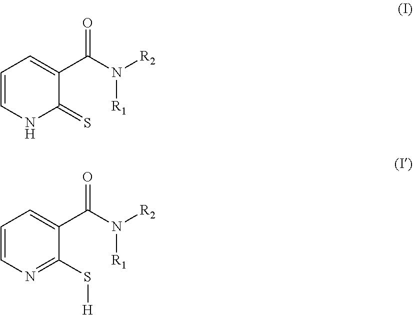

In which Formulas (I) and (I′):
R₁ denotes a radical chosen from:
   a) a hydrogen atom;
   b) a saturated linear C₁-C₁₀ or branched C₃-C₁₀ alkyl group optionally substituted with one or more groups, which may be identical or different, chosen from:
      i)—O—R₃
      ii)—S—R₃;
R₂ denotes a radical chosen from:
   a) a hydrogen atom;
   b) a saturated hydrocarbonated group linear C₁-C₁₂ or branched C₃-C₁₂ or cyclic C₃-C₈, optionally substituted with one or more groups, which may be identical or different, chosen from:
      i)—O—R₃
      ii)—S—R₃;
      iii)—C(O)—O—R₃;
      iv) a C₅-C₁₂ aryl group optionally substituted with one or more hydroxyls and/or with one or more C₁-C₈ alkoxy radicals;
   c) a C₅-C₁₂ aryl group optionally substituted with one or more hydroxyls and/or with one or more C₁-C₈ alkoxy radicals
R₃ denotes a radical chosen from:
   a) a hydrogen atom;
   b) a saturated linear C₁-C₁₀ or branched C₃-C₁₀ alkyl group;
      The compound (I′) is the tautomer form of the compound (I) when a tautomeric equilibrium exists according to the following scheme:

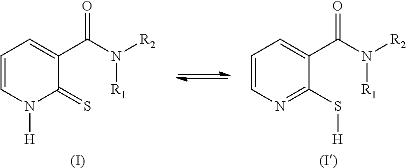

According to one embodiment of the disclosure R₁ represents one hydrogen atom.
According to another embodiment of the disclosure R₁ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably ethyl. Particularly the said alkyl group of R₁ is not substituted.
According to one embodiment of the disclosure R₂ represents one hydrogen atom.
According to another embodiment of the disclosure R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl group; the said alkyl group of R₂ being not substituted.
According to another embodiment of the disclosure R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl; the said alkyl group being substituted by one or more groups selected from i), ii), iii) and iv) as defined herein before. Preferably the said alkyl group being substituted by one or two groups selected from i), ii) and iii), more preferably by one or two groups selected from i) and iii), better substituted by one group iii) as carboxy.
Another variant for radical R₂ is that the said alkyl group being substituted by one group iv) especially substituted by one phenyl group.
According to another embodiment of the disclosure R₂ represents a (C₃-C₅)cycloalkyl group, preferably a (C₅-C₇)cycloalkyl group such cyclohexyl.
According to another embodiment of the disclosure R₂ represents C₅-C₁₂ aryl group optionally substituted with one or more hydroxyls and/or with one or more C₁-C₈ alkoxy radicals, preferably a phenyl group particularly not substituted.
According to an embodiment R₃ represents a hydrogen atom.
According to another embodiment R₃ represents a saturated linear C₁-C₁₀ or branched C₃-C₁₀ alkyl group; particularly a linear (C₁-C₆)alkyl group or a branched (C₃-C₆)alkyl group, preferably (C₁-C₄)alkyl group such as methyl group.
Preferably, the compounds of formula (I) and tautomer (I′) or their salts, their optical isomers, racemates, and/or solvates such as hydrates and the thereof, alone or as a mixture;
have the following meanings:
R₁ denotes a radical chosen from:
a) a hydrogen atom;
b) a saturated linear C₁-C₆ or branched C₃-C₆ alkyl group optionally substituted with one or more groups, which may be identical or different, chosen from:
   i)—O—R₃
   ii)—S—R₃;
   preferably optionally substituted with one or more groups i)
R₂ denotes a radical chosen from:
   a) a hydrogen atom;
   b) a saturated hydrocarbonated group linear C₁-C₁₀ or branched C₃-C₁₀ or cyclic C₃-C₈ such as C5-C6, optionally substituted with one or more groups, which may be identical or different, chosen from:
      i)—O—R₃
      ii)—S—R₃
      iii)—C(O)—O—R₃;
      iv) a phenyl group optionally substituted with one or more hydroxyls and/or with one or more C₁-C₄ alkoxy radicals such as methoxy;
      preferably substituted with one or more groups selected from i) and iii), preferably iii) such as carboxy
R₃ denotes a radical chosen from:
   a) a hydrogen atom;
   b) a saturated linear C₁-C₆ or branched C₃-C₆ alkyl group
      Preferentially, the compounds of formula (I) and tautomer (I′) or their salts, their optical isomers, racemates, and/or solvates such as hydrates and the thereof, alone or as a mixture;
      have the following meanings:
R₁ denotes a radical chosen from:
a) a hydrogen atom;
b) a saturated linear C₁-C₄ or branched C₃-C₄ alkyl group optionally substituted with one or more groups, which may be identical or different, chosen from i) —OR3, more preferably not substituted;
R₂ denotes a radical chosen from:
a) a hydrogen atom;
b) a saturated hydrocarbonated group linear C₁-C₁₀ or branched C₃-C₁₀ or cyclic C₃-C₈ as C5-C6, optionally substituted with one or more groups, which may be identical or different, chosen from:
   i)—O—R₃
   ii)—S—R₃;
   iv) a C₅-C₁₂ aryl group optionally substituted with one or more hydroxyls and/or with one or more C₁-C₄ alkoxy radicals;
R₃ denotes a radical chosen from:
a) a hydrogen atom;
b) a saturated linear C₁-C₄ or branched C₃-C₄ alkyl group such as methyl or ethyl.
   Preferentially, the compounds of formula (I) and tautomer (I′) or their salts, their optical isomers, racemates, and/or solvates such as hydrates and the thereof, alone or as a mixture;
   have the following meanings:
R₁ is a hydrogen atom; and
R₂ denotes a radical chosen from:
a) a hydrogen atom;
b) a saturated hydrocarbonated group linear C₁-C₅ or branched C₃-C₈ or cyclic C₃-C₈ as C5-C6, substituted with one or more groups, which may be identical or different, chosen from v) —C(O)—O—R₃, preferably substituted with one group iii) —C(O)—O—R₃;
R₂ is even more preferably a saturated hydrocarbonated group linear C₁-C₄ or branched C₃-C₄ substituted with one group iii) —C(O)—OR₃.
   According to another preferred embodiment, compounds of formula (I) and tautomer (I′) are selected among compounds of formula (II) and also the tautomers thereof, the salts thereof, the solvates thereof and the optical isomers thereof, and the racemates thereof, alone or as a mixture:

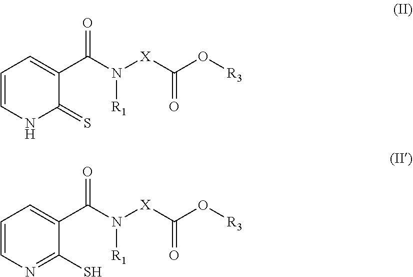

Formula (I) and (I′) Wherein R1 and R3 have the same meaning than for compounds of formula (I) and (I′) and X denotes an alkylene radical —(CH₂)ₙ— with n being an integer ranging inclusively from 1 to 10, preferably ranging from 1 to 6, more preferably ranging from 1 to 4, such as 1, preferably R₃ represents a hydrogen atom.
Among the compounds of formula (I), the following compounds are preferably used and their tautomer or their salts, their optical isomers, racemates, and/or solvates such as hydrates and the thereof, alone or as a mixture:

| No. | Structure | Chemical name | CAS No. |
|---|---|---|---|
| 1 | 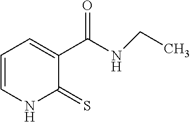 | N-ethyl-2-thioxo-1,2- dihydropyridine-3- carboxamide | 91859-75-5 |
| 2 | 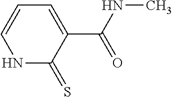 | N-methyl-2-thioxo- 1,2-dihydropyridine-3- carboxamide | 91859-74-4 |
| 3 | 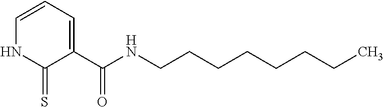 [SMILES: CCCCCCCCNC(=O)c1ccc[nH]c1=S] | N-octyl-2-thioxo-1,2- dihydropyridine-3- carboxamide | 91859-77-7 |
| 4 | 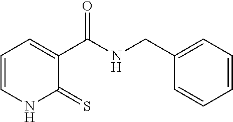 [SMILES: O=C(NCc1ccccc1)c1ccc[nH]c1=S] | N-benzyl-2-thioxo-1,2- dihydropyridine-3- carboxamide | 91859-79-9 |
| 5 | 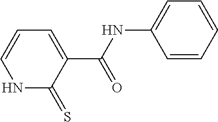 [SMILES: O=C(Nc1ccccc1)c1ccc[nH]c1=S] | N-phenyl-2-thioxo- 1,2-dihydropyridine-3- carboxamide | 104857-16-1 |
| 6 | 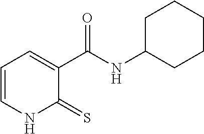 [SMILES: O=C(NC1CCCCC1)c1ccc[nH]c1=S] | N-cyclohexyl-2-thioxo- 1,2-dihydropyridine-3- carboxamide | 91859-78-8 |
| 7 | 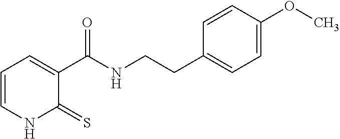 [SMILES: COc1ccc(CCNC(=O)c2ccc[nH]c2=S)cc1] | N-[2-(4- methoxyphenyl)ethyl]- 2-thioxo-1,2- dihydropyridine-3- carboxamide | 923682-88-6 |
| 8 | 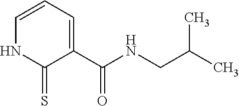 | N-(2-methylpropyl)-2- thioxo-1,2- dihydropyridine-3- carboxamide | 1100027-79-9 |
| 9 | 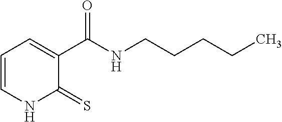 | N-pentyl-2-thioxo-1,2- dihydropyridine-3- carboxamide | 330667-57-7 |
| 10 | 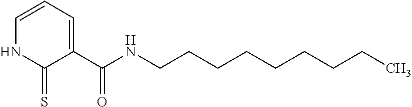 [SMILES: CCCCCCCCCNC(=O)c1ccc[nH]c1=S] | N-nonyl-2-thioxo-1,2- dihydropyridine-3- carboxamide | 1031149-44-6 |
| 11 | 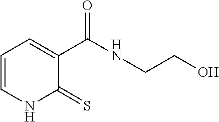 [SMILES: O=C(NCCO)c1ccc[nH]c1=S] | N-(2-hydroxyethyl)-2- thioxo-1,2- dihydropyridine-3- carboxamide | |
| 12 | 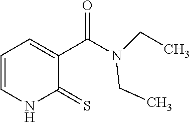 | N,N-diethyl 2- mercaptonicotinamide | |
| 13 | 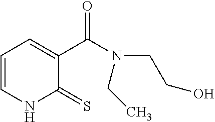 | N-ethyl-N-(2- hydroxyethyl)-2- thioxo-1,2- dihydropyridine-3- carboxamide | |
| 14 | 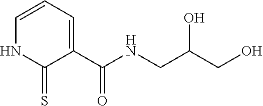 [SMILES: O=C(NCC(O)CO)c1ccc[nH]c1=S] | N-(2,3- dihydroxypropyl)-2- thioxo-1,2- dihydropyridine-3- carboxamide | |
| 15 | 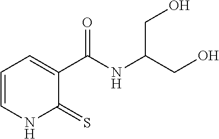 [SMILES: O=C(NC(CO)CO)c1ccc[nH]c1=S] | N-(1,3- dihydroxypropan-2- yl)-2-thioxo-1,2- dihydropyridine-3- carboxamide | |
| 16 | 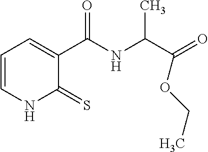 [SMILES: CCOC(=O)C(C)NC(=O)c1ccc[nH]c1=S] | Ethyl N-[(2-thioxo-1,2- dihydropyridin-3- yl)carbonyl]alaninate | |
| 17 | 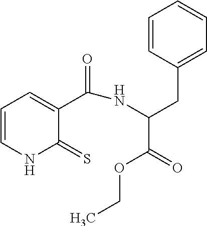 [SMILES: CCOC(=O)C(Cc1ccccc1)NC(=O)c1ccc[nH]c1=S] | Ethyl N-[(2-thioxo-1,2- dihydropyridin-3- yl)carbonyl]phenyl alaninate | |
| 18 | 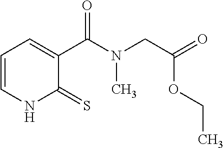 [SMILES: CCOC(=O)CN(C)C(=O)c1ccc[nH]c1=S] | Ethyl N-methyl-N-[(2- thioxo-1,2- dihydropyridin-3- yl)carbonyl]glycinate | |
| 19 | 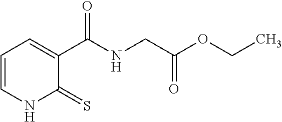 [SMILES: CCOC(=O)CNC(=O)c1ccc[nH]c1=S] | Ethyl N-[(2-thioxo-1,2- dihydropyridin-3- yl)carbonyl]glycinate | |
| 20 | 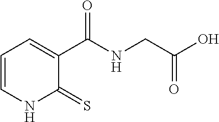 [SMILES: O=C(O)CNC(=O)c1ccc[nH]c1=S] | N-[(2-thioxo-1,2- dihydropyridin-3- yl)carbonyl]glycine | |
| 21 | 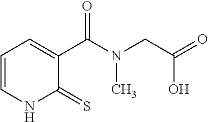 | N-methyl-N-[(2-thioxo- 1,2-dihydropyridin-3- yl)carbonyl]glycine | |
| 22 | 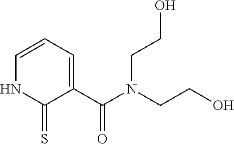 [SMILES: O=C(c1ccc[nH]c1=S)N(CCO)CCO] | N,N-bis(2- hydroxyethyl)-2- thioxo-1,2- dihydropyridine-3- carboxamide | |
| 23 | 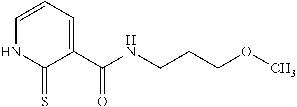 | N-(3-methoxypropyl)- 2-thioxo-1,2- dihydropyridine-3- carboxamide | |
| 24 | 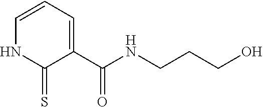 [SMILES: O=C(NCCCO)c1ccc[nH]c1=S] | N-butyl-2-thioxo-1,2- dihydropyridine-3- carboxamide | |

Among these compounds, the following compounds are more particularly preferred:

| No. | Structure | Chemical name | CAS No. |
|---|---|---|---|
| 1 | 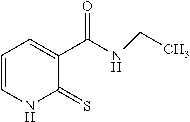 | N-ethyl-2-thioxo-1,2- dihydropyridine-3- carboxamide | 91859-75-5 |
| 2 | 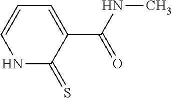 | N-methyl-2-thioxo- 1,2-dihydropyridine-3- carboxamide | 91859-74-4 |
| 4 | 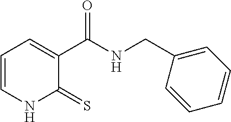 [SMILES: O=C(NCc1ccccc1)c1ccc[nH]c1=S] | N-benzyl-2-thioxo- 1,2-dihydropyridine-3- carboxamide | 91859-79-9 |
| 6 | 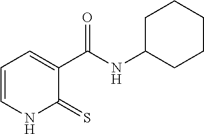 [SMILES: O=C(NC1CCCCC1)c1ccc[nH]c1=S] | N-cyclohexyl-2- thioxo-1,2- dihydropyridine-3- carboxamide | 91859-78-8 |
| 7 | 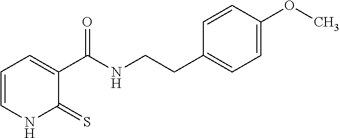 [SMILES: COc1ccc(CCNC(=O)c2ccc[nH]c2=S)cc1] | N-[2-(4- methoxyphenyl)ethyl]- 2-thioxo-1,2- dihydropyridine-3- carboxamide | 923682-88-6 |
| 9 | 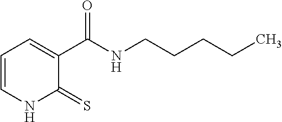 | N-pentyl-2-thioxo-1,2- dihydropyridine-3- carboxamide | 330667-57-7 |
| 11 | 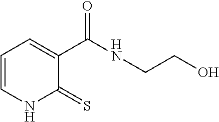 [SMILES: O=C(NCCO)c1ccc[nH]c1=S] | N-(2-hydroxyethyl)-2- thioxo-1,2- dihydropyridine-3- carboxamide | |
| 12 | 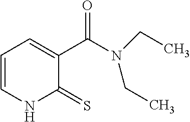 | N,N-diethyl 2- mercaptonicotinamide | |
| 14 | 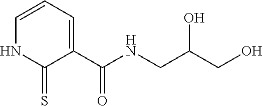 [SMILES: O=C(NCC(O)CO)c1ccc[nH]c1=S] | N-(2,3- dihydroxypropyl)-2- thioxo-1,2- dihydropyridine-3- carboxamide | |
| 15 | 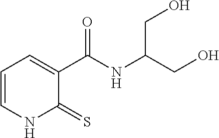 [SMILES: O=C(NC(CO)CO)c1ccc[nH]c1=S] | N-(1,3- dihydroxypropan-2- yl)-2-thioxo-1,2- dihydropyridine-3- carboxamide | |
| 16 | 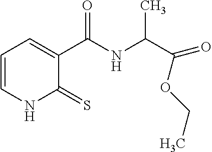 [SMILES: CCOC(=O)C(C)NC(=O)c1ccc[nH]c1=S] | Ethyl N-[(2-thioxo-1,2- dihydropyridin-3- yl)carbonyl]alaninate | |
| 17 | 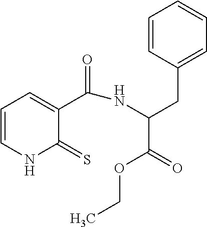 [SMILES: CCOC(=O)C(Cc1ccccc1)NC(=O)c1ccc[nH]c1=S] | Ethyl N-[(2-thioxo-1,2- dihydropyridin-3- yl)carbonyl]phenyl alaninate | |
| 18 | 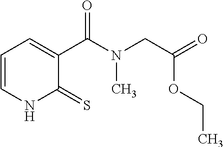 [SMILES: CCOC(=O)CN(C)C(=O)c1ccc[nH]c1=S] | Ethyl N-methyl-N-[(2- thioxo-1,2- dihydropyridin-3- yl)carbonyl]glycinate | |
| 19 | 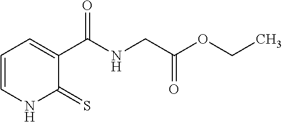 [SMILES: CCOC(=O)CNC(=O)c1ccc[nH]c1=S] | Ethyl N-[(2-thioxo-1,2- dihydropyridin-3- yl)carbonyl]glycinate | |
| 20 | 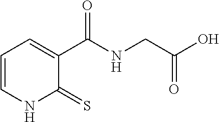 [SMILES: O=C(O)CNC(=O)c1ccc[nH]c1=S] | N-[(2-thioxo-1,2- dihydropyridin-3- yl)carbonyl]glycine | |
| 21 | 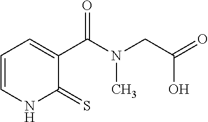 | N-methyl-N-[(2- thioxo-1,2- dihydropyridin-3- yl)carbonyl]glycine | |

More preferably, among these compounds, the following compounds are more particularly preferred:

| No. | Structure | Chemical name | CAS No. |
|---|---|---|---|
| 1 | 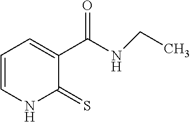 | N-ethyl-2-thioxo-1,2- dihydropyridine-3- carboxamide | 91859-75-5 |
| 9 | 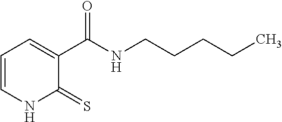 | N-pentyl-2-thioxo- 1,2-dihydropyridine- 3-carboxamide | 330667-57-7 |
| 16 | 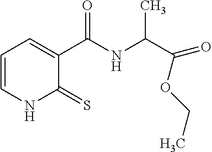 [SMILES: CCOC(=O)C(C)NC(=O)c1ccc[nH]c1=S] | Ethyl N-[(2-thioxo- 1,2-dihydropyridin-3- yl)carbonyl]alaninate | |
| 18 | 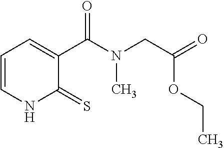 [SMILES: CCOC(=O)CN(C)C(=O)c1ccc[nH]c1=S] | Ethyl N-methyl-N-[(2- thioxo-1,2- dihydropyridin-3- yl)carbonyl]glycinate | |
| 19 | 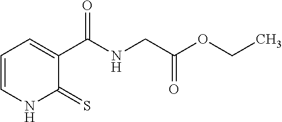 [SMILES: CCOC(=O)CNC(=O)c1ccc[nH]c1=S] | Ethyl N-[(2-thioxo- 1,2-dihydropyridin-3- yl)carbonyl]glycinate | |
| 20 | 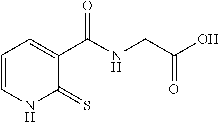 [SMILES: O=C(O)CNC(=O)c1ccc[nH]c1=S] | N-[(2-thioxo-1,2- dihydropyridin-3- yl)carbonyl]glycine | |
| 21 | 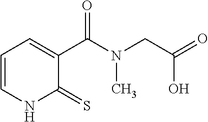 | N-methyl-N-[(2- thioxo-1,2- dihydropyridin-3- yl)carbonyl]glycine | |

Even more preferably, among these compounds, the following compounds are more particularly preferred:

| No. | Structure | Chemical name | CAS No. |
|---|---|---|---|
| 18 | 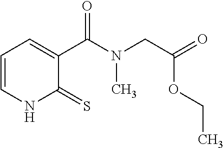 [SMILES: CCOC(=O)CN(C)C(=O)c1ccc[nH]c1=S] | Ethyl N-methyl-N-[(2- thioxo-1,2- dihydropyridin-3- yl)carbonyl]glycinate | |
| 19 | 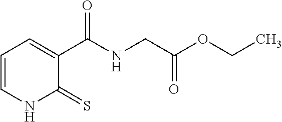 [SMILES: CCOC(=O)CNC(=O)c1ccc[nH]c1=S] | Ethyl N-[(2-thioxo- 1,2-dihydropyridin-3- yl)carbonyl]glycinate | |
| 20 | 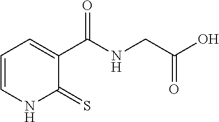 [SMILES: O=C(O)CNC(=O)c1ccc[nH]c1=S] | N-[(2-thioxo-1,2- dihydropyridin-3- yl)carbonyl]glycine | |
| 21 | 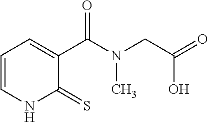 | N-methyl-N-[(2- thioxo-1,2- dihydropyridin-3- yl)carbonyl]glycine | |

In a most preferred embodiment, the compound according to the instant disclosure is the following:

| | | |
|---|---|---|
| 20 | 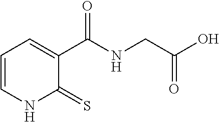 [SMILES: O=C(O)CNC(=O)c1ccc[nH]c1=S] | N-[(2-thioxo-1,2- dihydropyridin-3- yl)carbonyl]glycine |

All compounds can be obtained by chemical method known by man skilled in the art, from commercially available reagents. We can for example use the synthetic method disclosed in the European patent application EP3 390 363.
The composition used according to the disclosure comprises at least one compound of formulae (I) and/or (II) as described above, in a physiologically acceptable medium.
The compound (I), (I′), (II) and/or (II′) may be present in the composition used according to the disclosure in an amount which may be between 0.01% and 10% by weight, preferably between 0.1% and 5% by weight, in particular from 0.5% to 3% by weight, relative to the total weight of the composition.

### Hydrophilic Antioxidants

The one or more hydrophilic antioxidants may be selected from the group consisting of polyphenols and esters thereof; ascorbic acid (vitamin C) and esters thereof; and pharmaceutically acceptable salts thereof. The polyphenols are typically catechins; leucoanthocyanidins and flavanones; flavanins, flavones and anthocyanins; flavonols; flavonolignans; and oligomers thereof.

Ascorbic Acid and its Derivatives

The cosmetic compositions include ascorbic acid and its derivatives.

The composition according to the disclosure includes ascorbic acid or one of its derivatives, in a maximum content of 20% by weight relative to the total weight of composition.

Ascorbic acid within the meaning of the disclosure preferably corresponds to L-ascorbic acid, or vitamin C. Its structure is formula (A):

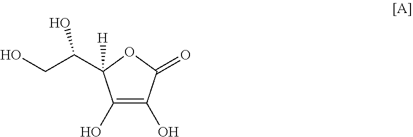 [SMILES: O=C1O[C@H]([C@@H](O)CO)C(O)=C1O]

By “ascorbic acid derivative” is preferably meant a compound selected from 5,6-di-O-dimethylsilylascorbate (in particular sold by the company Exsymol under the reference PRO-AA), the potassium salt of DL-alpha-tocopheryl-DL-ascorbyl-phosphate also called Potassium Ascorbyl Tocopheryl Phosphate (sold by Senju Pharmaceutical under the reference SEPIVITAL EPC), magnesium ascorbyl phosphate, sodium ascorbyl phosphate (sold by Roche company under the reference Stay-C 50), disodium ascorbyl sulfate, Ascorbyl palmitate, Tetrahexadecyl ascorbate, Ascorbyl tetraisopalmitate, 3-O-Ethyl ascorbic acid, sulfinyl-L-ascorbic acid, glucopyranosyl-L-ascorbic acid and ascorbyl glucoside.

By “ascorbyl glucoside” is meant the condensation product of glucose, in the form D, that is to say in the form of glucopyrannose α or β or furannosis α or β, or in the form L, with the ascorbic acid, preferably in the form L. Preferably, ascorbyl glucoside is le 2-O-α-D-glucopyranoside of L-ascorbic acid, in particular available from the company HAYASHIBARA.

Preferably, the composition according to the invention comprises ascorbic acid, preferably L-ascorbic acid.

The composition preferably comprises from 0.1% to 20 wt. % of ascorbic acid or one of its derivatives, based to the total weight of the composition, preferably from 1% to 15 wt. % based to the total weight of the composition.

The total amount of ascorbic acid and its derivatives may be present from about 0.1, 0.5, 1, 1.5, 2, 2.5, 3, 3.5, 4, 4.5, 5, 5.5, 6, 6.5, 7, 7.5, 8, 8.5, 9, 9.5 to about 9.5, 10, 10.5, 11, 11.5, 12, 12.5, 13, 13.5, 14, 14.5, 15, 15.5, 16, 16.5, 17, 17.5, 18, 18.5, 19, 19.5, or 20 wt. % based on the total weight of the composition.

### pH Adjusting Agent

The composition according to the present invention may comprise at least one pH adjusting agent (pH adjuster). Two or more pH adjusting agents may be used in combination. Thus, a single type of pH adjusting agent or a combination of different types of pH adjusting agents may be used.

As the pH adjusting agent, at least one acidifying agent and/or at least one basifying agent (alkaline agent) may be used.

The acidifying agent may be a monovalent or polyvalent, such as divalent, acid.

The acidifying agents can be, for example, mineral (inorganic) acids such as hydrochloric acid, sulfuric acid, phosphoric acid, or organic acids such as carboxylic acids, for instance tartaric acid, citric acid, and lactic acid, as well as sulphonic acids.

The basifying agent may be a monovalent or polyvalent, such as divalent, base. The basifying agents may be mineral (inorganic) or organic, or hybrid.

The mineral basifying agents may be chosen from aqueous ammonia; alkali metal carbonates or bicarbonates such as sodium or potassium carbonates and sodium or potassium bicarbonates; alkali metal hydroxides such as sodium hydroxide and potassium hydroxide; and mixtures thereof.

The organic basifying agents may be chosen from organic amines with a pKb at 25° C. of less than 12, preferably less than 10, and even more advantageously less than 6. It should be noted that it is the pKb corresponding to the function of highest basicity. In addition, the organic amines do not comprise any alkyl or alkenyl fatty chains comprising more than ten carbon atoms.

The organic basifying agent may be chosen, for example, from alkanolamines, oxyethylenated and/or oxypropylenated ethylenediamines, amino acids and amine compounds of formula (III) below:

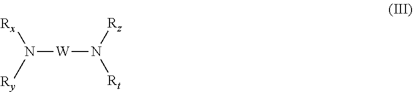

in which
W represents a C₁-C₆ divalent alkylene radical optionally substituted with one or more hydroxyl groups or a C₁-C₆ alkyl radical, and optionally interrupted with one or more heteroatoms such as O and N, and
Rₓ, R_{y}, R_{z}, and Rₜ, which may be identical or different, represent a hydrogen atom or a C₁-C₆ alkyl, C₁-C₆ hydroxyalkyl, or C₁-C₆ aminoalkyl radical.
   Examples of the amine compounds of formula (III) that may be mentioned include 1,3-diaminopropane, 1,3-diamino-2-propanol, spermine and spermidine.
   The term “alkanolamine” means an organic amine comprising a primary, secondary or tertiary amine function, and one or more linear or branched C₁-C₈ alkyl groups bearing one or more hydroxyl radicals.
   Alkanolamines such as monoalkanolamines, dialkanolamines or trialkanolamines comprising one to three identical or different C₁-C₄ hydroxyalkyl radicals may be suitable for the present invention. Among the compounds of this type, mention may be made of monoethanolamine (MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.
   Amino acids that may be used are of natural or synthetic origin, in their L, D or racemic form, and comprise at least one acid function chosen more particularly from carboxylic acid, sulfonic acid, phosphonic acid or phosphoric acid functions. The amino acids may be in neutral or ionic form.
   As amino acids that may be used in the present invention, mention may be made especially of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.
   It may be preferable that the amino acids are basic amino acids comprising an additional amine function optionally included in a ring or in an ureido function.
   Such basic amino acids may preferably be chosen from those corresponding to formula (IV) below:

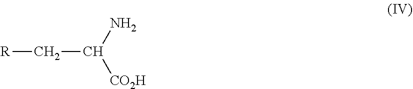

in which
R represents a group chosen from:

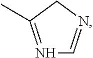

—O—R₃,
—S—R₃,
—C(O)—O—R₃,

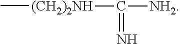

The compounds corresponding to formula (IV) include histidine, lysine, arginine, ornithine and citrulline.
The organic basifying agent may be chosen from organic amines of heterocyclic type. Besides histidine that has already been mentioned in the amino acids, mention may in particular be made of pyridine, piperidine, imidazole, triazole, tetrazole and benzimidazole.
The organic basifying agent may also be chosen from amino acid dipeptides. As amino acid dipeptides that may be used in the present invention, mention may be made especially of carnosine, anserine and baleine.
The organic basifying agent may also be chosen from compounds comprising a guanidine function. As amines of this type that may be used in the present invention, besides arginine, which has already been mentioned as an amino acid, mention may be made especially of creatine, creatinine, 1,1-dimethylguanidine, 1,1-diethyl-guanidine, glycocyamine, metformin, agmatine, N-amidinoalanine, 3-guanidino-propionic acid, 4-guanidinobutyric acid and 2-([amino(imino)methyl]amino)ethane-1-sulfonic acid.
In a preferred embodiment of the present invention, the organic basifying agent may be selected from amino acids, preferably basic amino acids, and more preferably arginine, lysine, histidine or mixtures thereof. Even more preferentially, the organic basifying agent may be arginine.
Hybrid compounds that may be mentioned include the salts of the amines mentioned previously with acids such as carbonic acid or hydrochloric acid. Guanidine carbonate or monoethanolamine hydrochloride may be used in particular.
The pH adjusting agent may be present in an amount of 0.01% by weight or more, preferably 0.05% by weight or more, and more preferably 0.1% by weight or more, relative to the total weight of the composition.
The pH adjusting agent may be present in an amount of 15% by weight or less, preferably 10% by weight or less, and more preferably 5% by weight or less, relative to the total weight of the composition.
The pH adjusting agent may be present in an amount ranging from 0.01% to 15% by weight, preferably from 0.05% to 10% by weight, and more preferably from 0.1% to 5% by weight or less, relative to the total weight of the composition.
It is preferable that the composition according to the present invention have a pH of 4.5 or more, and more preferably 5 or more.
It is preferable that the composition according to the present invention have a pH of 6.5 or less, and more preferably 6 or less.
It is preferable that the composition according to the present invention have a pH of from 4.5 to 6.5, and more preferably from 5 to 6.
The pH of the composition means the pH of the aqueous phase of the composition according to the present invention.
It may be preferable that at least one buffer or buffering agent also be used, as the pH adjusting agent, in combination with the acidifying agent and/or the basifying agent, in order to stabilize the pH of the composition according to the present invention.
As the buffer, any of commonly known buffers may be used. For example, salts of acids or bases, preferably salts of weak acids or weak bases, may be used. For example, sodium citrate or sodium lactate may be used as the buffer, if citric acid or lactic acid is used as the acidifying agent.

### Cosmetically Acceptable Carrier System

The cosmetic compositions include a cosmetically acceptable carrier system. The term “cosmetically acceptable” means a carrier that is compatible with any keratinous substrate, and for purposes hereof, includes water and optionally water based solvents subject to any exclusions as disclosed herein.

The cosmetic compositions may comprise any constituent normally employed in the topical application and administration envisaged. Mention may in particular be made of water, solvents, polyols, fatty compounds (i.e. described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety), pigments, fillers, silicones, surfactants, thickeners, gelling agents, preservatives, chelating agents, and their mixtures in all proportions.

The composition used according to the invention may then comprise any adjuvant commonly used in the cosmetics field.

Methods of Use

The instant disclosure also relates to methods of using the cosmetic compositions described herein. For example, the cosmetic compositions can be used in a method that comprises applying the cosmetic compositions to the skin of humans. In some cases, the composition is applied to the face. Furthermore, the cosmetic composition can be used in methods for depigmenting, lightening and/or bleaching keratin materials, preferably skin, comprising the step of: applying to the keratin substance the composition according to the compositions described herein. The aforementioned methods are non-therapeutic.

The cosmetic composition may be applied once per day, twice per day, or more than once or twice per day. In some cases, the composition is applied in the evenings before bed. In other cases, the compositions are applied in the morning. In still other cases, the composition may be applied immediately after washing the skin. The compositions may be used once, or for a series of days, weeks, or months. For example, the compositions may be used daily for a period of 1, 2, 3, 4, 5, 6, 7, 8 or more weeks, or months.

The instant disclosure also relates to non-therapeutic cosmetic process for depigmenting, lightening and/or bleaching keratin materials, preferably skin, comprising the step of applying to the keratin substance the composition according to the compositions described herein.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### EXAMPLES

Implementation of the present disclosure is provided by way of the following examples. The following examples serve to elucidate aspects of the technology without being limiting in nature.

### Example 1

### Example 1: Synthesis of Compound 20

Compound 20 is synthesized as disclosed in example 2 of patent EP3 390 363.

### Example 2

### Inventive Compositions

TABLE 1
Inventive Compositions

| | | | Inven- | Inven- |
|---|---|---|---|---|
| | | | tive | tive |
| | Ingredients | INCI US | Ex. 1 | Ex. 2 |
| a | | Compound of | 0.50 | 0.50 |
| | | thiopyridinone type (THP) | | |
| b | | Ascorbic Acid | 10.00 | 10.00 |
| | Fatty compounds | Fatty compounds | 10 | 10 |
| | Polymers | Polymers | 1.5 | 1.5 |
| | Emulsifiers | Emulsifiers | 2.5 | 2.5 |
| | Filler | Filler | 2.0 | 2.0 |
| | Chelating agent | Chelating agent | 0.11 | 0.11 |
| | Active Compounds | Active compounds | 1.0 | 1.0 |
| | Vegetal Extract | Vegetal Extract | | 0.50 |
| | Preservative | Preservative | 0.80 | 0.80 |
| | Solvent | Solvents | 6.21 | 6.21 |
| | pH adjuster | pH adjuster | 2.20 | 2.20 |
| | Q.S. | Water | 62.58 | 62.07 |

### Example 3

Inventive Examples 1 and 2 and several comparative Examples were tested for stability. The stability of the inventive and comparative Examples was assessed by following the procedure described below.

Formula bulk was added to a 30 mL aluminum tube that was then crimped and sealed. An initial, freshly made sample (TO) was submitted for Thiopyridone (THP) metering. Samples were placed in temperature-controlled ovens at 25° C. and 45° C. for a total of 2 months. At 1 month and 2 months at each temperature, samples were removed and submitted for Thiopyridone (THP) metering. The % remaining presented in Table 2 compares the 2 months, 45° C. sample to the initial TO sample and is presented as % of THP remaining in sample when compared to T0. Metering is performed via high performance liquid chromatography with a UV detector.

The results are presented in Table 2 below.

TABLE 2
Stability Results

| | | Vitamin C | | | | | |
|---|---|---|---|---|---|---|---|
| | Examples | Initial | T1M 25 | T1M 45 | T2M 25 | T2M 45 | % Remaining |
| Vit C only | Comp. Ex. 1 | 11.30% | 9.93% | 9.37% | 9.84% | 8.82% | 78.05% |
| | Comp. Ex. 2 | 11.16% | 10.15% | 9.43% | 9.66% | 9.00% | 80.65% |
| | Comp. Ex. 3 | 10.94% | 10.22% | 9.50% | 9.95% | 9.50% | 86.84% |
| | Comp. Ex. 4 | 10.53% | 9.82% | 9.16% | 10% | 8.97% | 85.19% |
| | Comp. Ex. 5 | 10.54% | 9.82% | 8.97% | 10.11% | 8.62% | 81.78% |
| | Comp. Ex. 6 | 11.01% | 9.91% | Tube burst | 10.36% | Tube burst | Tube burst |
| Vit C + THP | Inventive Ex. 1 | 8.70% | 9.45% | 9.06% | 9.76% | 8.98% | ~100% |
| | Inventive Ex. 2 | 9.72% | 9.01% | 8.71% | 9.67% | 8.89% | 91.46% |

| | | |
|---|---|---|
| THP Only | Comp. Ex. 7 | N/A |

| | | THP | | | | | |
|---|---|---|---|---|---|---|---|
| | Examples | Initial | T1M 25 | T1M 45 | T2M 25 | T2M 45 | % Remaining |
| Vit C only | Comp. Ex. 1 | N/A | | | | | |
| | Comp. Ex. 2 | | | | | | |
| | Comp. Ex. 3 | | | | | | |
| | Comp. Ex. 4 | | | | | | |
| | Comp. Ex. 5 | | | | | | |
| | Comp. Ex. 6 | | | | | | |
| Vit C + THP | Inventive Ex. 1 | 0.47% | 0.46% | 0.45% | 0.50% | 0.49% | ~100% |
| | Inventive Ex. 2 | 0.48% | 0.47% | 0.46% | 0.50% | 0.49% | ~100% |
| THP Only | Comp. Ex. 7 | 0.512% | 0.493% | 0.461% | 0.466% | 0.418% | 81.64% |

As shown above, Inventive Examples 1 and 2, containing both, Vitamin C and THP (=compound of Thiopyridinone type), demonstrated that the chemical stability of THP (=compound of Thiopyridinone type) and Vitamin C increased when the two molecules are associated. They showed between 91% through 100% of stability for both actives. There was also an improved efficacy with the association of Vitamin C and THP (=compound of Thiopyridinone type). The higher stability lead to a higher overall active concentration when the consumer received and used the product. For example, if the formula contains 10% of Vit C alone and it is unstable, if there is 40% degradation of Vit C, now there will only be 6% Vit C left. However, if there is combination of Vit C and THP (=compound of Thiopyridinone type), and Vit C barely degrades, the Vit C will still be about 10% Vit C (i.e., 10% Vit C will be more efficacious than 6% Vit C).

Comparative Examples 1, 2, 3, 4, 5 and 6 containing only Vitamin C showed between 78% to 86% stability.

Comparative Example 7 containing only compound of Thiopyridinone type demonstrated that the association of Vit C and THP improve the stability of the THP when it is in combination with the Vit C than when it is alone.

The data show that the cosmetic compositions according to the instant disclosure, including both Vitamin C and THP (=compound of Thiopyridinone type), are surprisingly stable.

While the disclosure has been described with reference to described embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The term “INCI” is an abbreviation of International Nomenclature of Cosmetic Ingredients, which is a system of names provided by the International Nomenclature Committee of the Personal Care Products Council to describe personal care ingredients.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt %” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
i) at least one compound selected from compounds of formula (I) and tautomer of formula (I′) herein below or their salts; and their optical isomers, racemates, and/or solvates such as hydrates, alone or as a mixture: 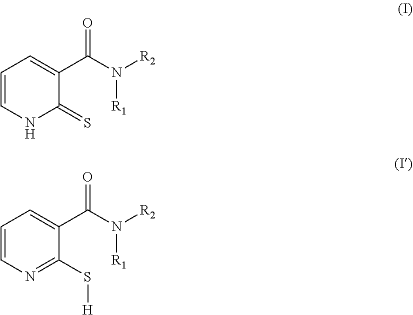
In which Formulas (I) and (I′):
R₁ denotes a radical chosen from:
a) a hydrogen atom;
b) a saturated linear C₁-C₁₀ or branched C₃-C₁₀ alkyl group optionally substituted with one or more groups, which may be identical or different, chosen from:
i)—O—R₃
ii)—S—R₃;
R₂ denotes a radical chosen from:
a) a hydrogen atom;
b) a saturated hydrocarbonated group linear C₁-C₁₂ or branched C₃-C₁₂ or cyclic C₃-C₈, optionally substituted with one or more groups, which may be identical or different, chosen from:
i)—O—R₃
ii)—S—R₃;
iii)—C(O)—O—R₃;
iv) a C₅-C₁₂ aryl group optionally substituted with one or more hydroxyls and/or with one or more C₁-C₈ alkoxy radicals;
c) a C₅-C₁₂ aryl group optionally substituted with one or more hydroxyls and/or with one or more C₁-C₈ alkoxy radicals
R₃ denotes a radical chosen from:
a) a hydrogen atom;
b) a saturated linear C₁-C₁₀ or branched C₃-C₁₀ alkyl group;
ii) one or more hydrophilic antioxidants.

2. The composition of claim 1 in which radical:
R₁ of formula (I) and (I′) represents a hydrogen atom,
or
R₁ of formula (I) and (I′) represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably ethyl, particularly the said alkyl group of R₁ is not substituted.

3. The composition of claim 1 in which:
R₂ of formula (I) and (I′) represents a hydrogen atom;
or
R₂ of formula (I) and (I′) represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl group; the said alkyl group of R₂ being not substituted.

4. The composition of claim 1 in which radical:
R₂ of formula (I) and (I′) represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl; the said alkyl group being substituted by one or more groups selected from i), ii), iii) and iv) as defined in claim 1, preferably the said alkyl group being substituted by one or two groups selected from i), ii) and iii), more preferably by one or two groups selected from i) and iii), better substituted by one group iii) as carboxy.

5. The composition of claim 1 in which radical:
R₂ of formula (I) and (I′) represents a (C₃-C₅)cycloalkyl group, preferably a (C₅-C₇)cycloalkyl group such cyclohexyl;
or
R₂ of formula (I) and (I′) represents a C₅-C₁₂ aryl group optionally substituted with one or more hydroxyls and/or with one or more C₁-C₈ alkoxy radicals, preferably a phenyl group particularly not substituted.

6. The composition of claim 1 in which:
R₃ of formula (I) and (I′) represents a hydrogen atom;
or
R₃ of formula (I) and (I′) represents a saturated linear C₁-C₁₀ or branched C₃-C₁₀ alkyl group; particularly a linear (C₁-C₆)alkyl group or a branched (C₃-C₆)alkyl group, preferably (C₁-C₄)alkyl group such as methyl group.

7. The composition of claim 1 in which:
R₁ of formula (I) and (I′) represents a radical chosen from:
a) a hydrogen atom;
b) a saturated linear C₁-C₆ or branched C₃-C₆ alkyl group optionally substituted with one or more groups, which may be identical or different, chosen from:
i)—O—R₃
ii)—S—R₃;
preferably optionally substituted with one or more groups i)
R₂ of formula (I) and (I′) represents a radical chosen from:
a) a hydrogen atom;
b) a saturated hydrocarbonated group linear C₁-C₁₀ or branched C₃-C₁₀ or cyclic C₃-C₈ such as C5-C6, optionally substituted with one or more groups, which may be identical or different, chosen from:
i)—O—R₃
ii)—S—R₃;
iii)—C(O)—O—R₃;
iv) a phenyl group optionally substituted with one or more hydroxyls and/or with one or more C₁-C₄ alkoxy radicals such as methoxy;
preferably substituted with one or more groups selected from i) and iii), preferably iii) such as carboxy
R₃ of formula (I) and (I′) represents a radical chosen from:
a) a hydrogen atom;
b) a saturated linear C₁-C₆ or branched C₃-C₆ alkyl group;
preferentially, the compounds of formula (I) and tautomer (I′) or their salts, their optical isomers, racemates, and/or solvates such as hydrates and the thereof, alone or as a mixture;
have the following meanings:
R₁ of formula (I) and (I′) represents a radical chosen from:
a) a hydrogen atom;
b) a saturated linear C₁-C₄ or branched C₃-C₄ alkyl group optionally substituted with one or more groups, which may be identical or different, chosen from i) —OR₃, more preferably not substituted;
R₂ of formula (I) and (I′) represents a radical chosen from:
a) a hydrogen atom;
b) a saturated hydrocarbonated group linear C₁-C₁₀ or branched C₃-C₁₀ or cyclic C₃-C₈ as C5-C6, optionally substituted with one or more groups, which may be identical or different, chosen from:
i)—O—R₃
ii)—S—R₃;
iv) a C₅-C₁₂ aryl group optionally substituted with one or more hydroxyls and/or with one or more C₁-C₄ alkoxy radicals;
R₃ of formula (I) and (I′) represents a radical chosen from:
a) a hydrogen atom;
b) a saturated linear C₁-C₄ or branched C₃-C₄ alkyl group such as methyl or ethyl.

8. The composition of claim 1 in which compounds of formula (I), the compounds 1 to 24 and their tautomer or their salts, their optical isomers, racemates, and/or solvates such as hydrates, alone or as a mixture, particularly compounds 1, 2, 4, 6, 7, 9, 11, 12, 14, 15, 16, 17, 18, 19, 20, or 21, more particularly 1, 9, 16, 18, 19, 20, or 21, preferably 18, 19, 20, or 21, more preferably 20:

| No. | Structure | Chemical name |
|---|---|---|
| 1 | 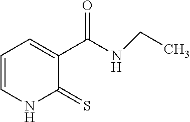 | N-ethyl-2-thioxo-1,2-dihydropyridine- 3-carboxamide |
| 2 | 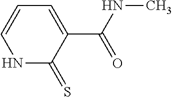 | N-methyl-2-thioxo-1,2- dihydropyridine-3-carboxamide |
| 3 | 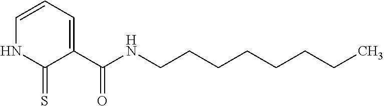 [SMILES: CCCCCCCCNC(=O)c1ccc[nH]c1=S] | N-octyl-2-thioxo-1,2-dihydropyridine- 3-carboxamide |
| 4 | 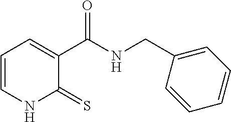 [SMILES: O=C(NCc1ccccc1)c1ccc[nH]c1=S] | N-benzyl-2-thioxo-1,2- dihydropyridine-3-carboxamide |
| 5 | 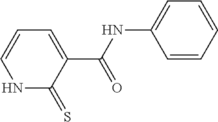 [SMILES: O=C(Nc1ccccc1)c1ccc[nH]c1=S] | N-phenyl-2-thioxo-1,2- dihydropyridine-3-carboxamide |
| 6 | 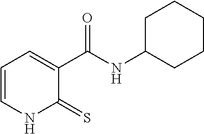 [SMILES: O=C(NC1CCCCC1)c1ccc[nH]c1=S] | N-cyclohexyl-2-thioxo-1,2- dihydropyridine-3-carboxamide |
| 7 | 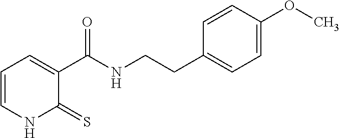 [SMILES: COc1ccc(CCNC(=O)c2ccc[nH]c2=S)cc1] | N-[2-(4-methoxyphenyl)ethyl]-2- thioxo-1,2-dihydropyridine-3- carboxamide |
| 8 | 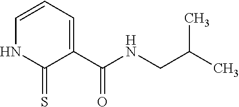 | N-(2-methylpropyl)-2-thioxo-1,2- dihydropyridine-3-carboxamide |
| 9 | 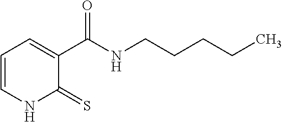 | N-pentyl-2-thioxo-1,2- dihydropyridine-3-carboxamide |
| 10 | 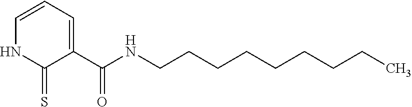 [SMILES: CCCCCCCCCNC(=O)c1ccc[nH]c1=S] | N-nonyl-2-thioxo-1,2- dihydropyridine-3-carboxamide |
| 11 | 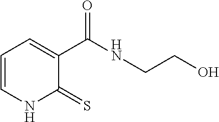 [SMILES: O=C(NCCO)c1ccc[nH]c1=S] | N-(2-hydroxyethyl)-2-thioxo-1,2- dihydropyridine-3-carboxamide |
| 12 | 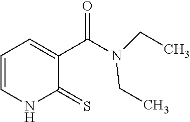 | N,N-diethyl 2-mercaptonicotinamide |
| 13 | 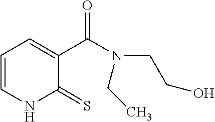 | N-ethyl-N-(2-hydroxyethyl)-2-thioxo- 1,2-dihydropyridine-3-carboxamide |
| 14 | 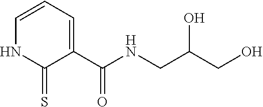 [SMILES: O=C(NCC(O)CO)c1ccc[nH]c1=S] | N-(2,3-dihydroxypropyl)-2-thioxo-1,2- dihydropyridine-3-carboxamide |
| 15 | 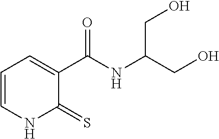 [SMILES: O=C(NC(CO)CO)c1ccc[nH]c1=S] | N-(1,3-dihydroxypropan-2-yl)-2- thioxo-1,2-dihydropyridine-3- carboxamide |
| 16 | 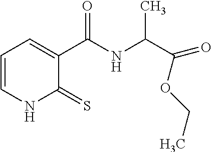 [SMILES: CCOC(=O)C(C)NC(=O)c1ccc[nH]c1=S] | Ethyl N-[(2-thioxo-1,2- dihydropyridin-3- yl)carbonyl]alaninate |
| 17 | 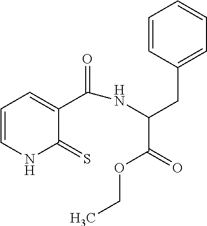 [SMILES: CCOC(=O)C(Cc1ccccc1)NC(=O)c1ccc[nH]c1=S] | Ethyl N-[(2-thioxo-1,2- dihydropyridin-3-yl)carbonyl]phenyl alaninate |
| 18 | 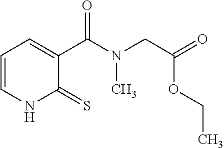 [SMILES: CCOC(=O)CN(C)C(=O)c1ccc[nH]c1=S] | Ethyl N-methyl-N-[(2-thioxo-1,2- dihydropyridin-3- yl)carbonyl]glycinate |
| 19 | 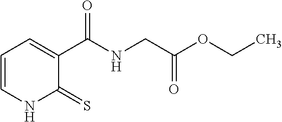 [SMILES: CCOC(=O)CNC(=O)c1ccc[nH]c1=S] | Ethyl N-[(2-thioxo-1,2- dihydropyridin-3- yl)carbonyl]glycinate |
| 20 | 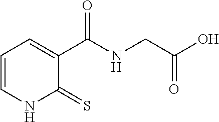 [SMILES: O=C(O)CNC(=O)c1ccc[nH]c1=S] | N-[(2-thioxo-1,2-dihydropyridin-3- yl)carbonyl]glycine |
| 21 | 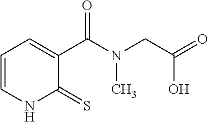 | N-methyl-N-[(2-thioxo-1,2- dihydropyridin-3-yl)carbonyl]glycine |
| 22 | 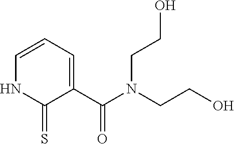 [SMILES: O=C(c1ccc[nH]c1=S)N(CCO)CCO] | N,N-bis(2-hydroxyethyl)-2-thioxo-1,2- dihydropyridine-3-carboxamide |
| 23 | 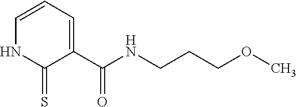 | N-(3-methoxypropyl)-2-thioxo-1,2- dihydropyridine-3-carboxamide |
| 24 | 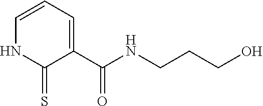 [SMILES: O=C(NCCCO)c1ccc[nH]c1=S] | N-butyl-2-thioxo-1,2-dihydropyridine- 3-carboxamide |

9. The cosmetic composition of claim 1, wherein the i) compound of thiopyridinone type and the ii) one or more hydrophilic antioxidant are present in the composition in amounts sufficient to produce stability of i) and ii).

10. The composition of claim 1 in which the compound ii) represents one or more ascorbic acid and its derivatives.

11. The composition of claim 11, wherein the ii) one or more ascorbic acid and its derivatives is present from about 0.1 to about 20 wt. %, based on the total weight of the cosmetic composition.

12. The composition of claim 1, wherein the one or more ascorbic acid and its derivatives is present from about 1 to about 15 wt. %, based on the total weight of the cosmetic composition.

13. The composition of claim 1, wherein the i) compound of thiopyridininone type is present from about 0.01 to about 10 wt. %, based on the total weight of the cosmetic composition.

14. The composition of claim 1, wherein the i) compound of thiopyridininone type is present from about 0.1 to about 5 wt. %, based on the total weight of the cosmetic composition.

15. The composition of claim 1, wherein the pH is from about 4.5 to about 6.5, preferably from about 5 to about 6.

16. A cosmetic composition comprising:
i) from about 0.01 to about 10 wt. % of at least one compound selected from compounds of formula (I) and tautomer of formula (I′) herein below or their salts; and their optical isomers, racemates, and/or solvates such as hydrates, alone or as a mixture: 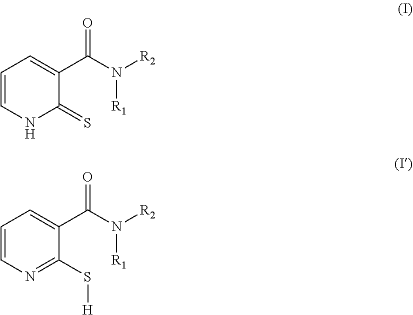
In which Formulas (I) and (I′):
R₁ denotes a radical chosen from:
a) a hydrogen atom;
b) a saturated linear C₁-C₁₀ or branched C₃-C₁₀ alkyl group optionally substituted with one or more groups, which may be identical or different, chosen from:
i)—O—R₃
ii)—S—R₃;
R₂ denotes a radical chosen from:
a) a hydrogen atom;
b) a saturated hydrocarbonated group linear C₁-C₁₂ or branched C₃-C₁₂ or cyclic C₃-C₈, optionally substituted with one or more groups, which may be identical or different, chosen from:
i)—O—R₃
ii)—S—R₃
iii)—C(O)—O—R₃;
iv) a C₅-C₁₂ aryl group optionally substituted with one or more hydroxyls and/or with one or more C₁-C₈ alkoxy radicals;
c) a C₅-C₁₂ aryl group optionally substituted with one or more hydroxyls and/or with one or more C₁-C₈ alkoxy radicals
R₃ denotes a radical chosen from:
a) a hydrogen atom;
b) a saturated linear C₁-C₁₀ or branched C₃-C₁₀ alkyl group;
ii) from about 0.1 to about 20 wt. % of one or more hydrophilic antioxidant; and
iii) a cosmetic suitable carrier;
wherein the i) compound of thiopyridininone type and the ii) one or more hydrophilic antioxidants are present in the composition in amounts sufficient to produce stability of i) and ii); and
wherein the weight percentages are based on the total weight of the cosmetic composition.

17. A method for stabilizing compound of thiopyridininone type and hydrophilic antioxidant when both present in a composition, in an amount sufficient to produce stability of compound of thiopyridininone type and hydrophilic antioxidant.

18. A method for treating skin comprising applying a cosmetic composition of claim 1 or 16 to the skin.

19. A non-therapeutic cosmetic process for depigmenting, lightening and/or bleaching keratin materials, preferably skin, comprising the step of: applying to the keratin substance the composition of claim 1 or 16.
